(19) United States
(12) Patent Application Publication
(10) Pub. No.: US 2011/0288181 A1
(43) Pub. Date: Nov. 24, 2011

(54) PREPARATIONS OF BIOLOGICALLY ACTIVE SUBSTANCES WITH ENLARGED SURFACE BASED ON AMPHIPHILIC COPOLYMERS

(75) Inventors: Sebastian Koltzenburg, Dannstadt-Schauernheim (DE); Karl Kolter, Limburgerhof (DE); Jan Kurt Walter Sandler, Heidelberg (DE); Dejan Djuric, Mannheim (DE); Ingo Bellin, Mannheim (DE)

(73) Assignee: BASF SE, Ludwigshafen (DE)

(21) Appl. No.: 13/112,193

(22) Filed: May 20, 2011

Related U.S. Application Data
(60) Provisional application No. 61/346,932, filed on May 21, 2010.

(51) Int. Cl. A61K 47/34; B29C 44/00; B29C 47/00; A01N 25/10

(52) U.S. Cl. 514/772.1; 264/50

(57) ABSTRACT

Preparations with enlarged surface comprising an active ingredient and an amphiphilc copolymer, wherein the copolymer comprises at least one polyether-containing graft polymer. The preparations are partially or completely foamed. Processes for producing the preparations are also described, including processes comprising extrusion of a melt impregnated with a physiologically acceptable volatile blowing agent.

### CROSS REFERENCE TO RELATED APPLICATIONS

This application claims the benefit of U.S. Provisional Application Ser. No. 61/346,932, filed May 21, 2010, which is hereby incorporated by reference.

### BACKGROUND OF THE INVENTION

It is well known that foamed plastics can be produced via extrusion of melts comprising volatile blowing agents. The conditions that can affect the morphology of the foams are also known.

By way of example, M. Lee et al. in Polymer Engineering and Science, Vol. 38, No. 7, 1998, describe the extrusion of foamed polyethylene/polystyrene blends with supercritical carbon dioxide.

Han et al. in “Polymer Engineering and Science, Vol. 42, No. 11, 2094-2106”, moreover describe the foam extrusion of polystyrenes with supercritical carbon dioxide.

W. Michaeli et al., ANTEC 2007/pages 3043-3045; Lee et al., Polym Int 49:712-718 (2000) are other publications relating to this topic.

WO 2007/051743 discloses the use of water-soluble or water-dispersible copolymers of N-vinyllactam, vinyl acetate, and polyethers as solubilizers for pharmaceutical, cosmetic, food-technology, agrotechnical, or other technical applications. That document very generally says that the corresponding graft polymers can also be processed in the melt with the active ingredients.

WO 2009/013202 discloses that these graft polymers of N-vinyllactam, vinyl acetate, and polyethers can be melted in an extruder and mixed with pulverulent or liquid active ingredients, where the extrusion process described takes place at temperatures markedly below the melting point of the active ingredient.

EP-A 0 932 393 discloses solid foamed drug forms obtained via extrusion and foaming of active-ingredient-containing polymer melts comprising active ingredients and thermoplastic polymers, such as homo- and copolymers of N-vinylpyrrolidone. These foamed drug forms are said to exhibit markedly improved active ingredient release, when compared with the unfoamed extrudates.

WO 2005/023215 describes active-ingredient-containing lamellar polymer particles, these being obtained via melt extrusion and foaming of the melt by using a gas. Polymers mentioned comprise poly(vinylpyrrolidone-vinyl acetate) or Eudragit E11-PO. The foamed lamellar particles are said to permit faster release of the active ingredient in an aqueous environment.

However, the foam formulations known hitherto for biologically active substances remain unsatisfactory with regard to the mechanical stability of the foams.

Furthermore, dissolution performance is still unsatisfactory in the case of preparations of active ingredients which are sparingly soluble in water.

### SUMMARY OF THE INVENTION

The present invention relates to solid preparations of active ingredients, such as biologically active substances, with enlarged surface based on thermoplastically processable amphiphilic copolymers, where the amphiphilic copolymer used comprises at least one polyether-containing graft polymer. The invention further relates to processes for producing these preparations.

The enlargement of the surface is achieved via partial or complete foaming of the preparations.

### DETAILED DESCRIPTION

It was an object of the present invention to find active-ingredient-containing preparations which can be obtained by the attractive process of melt extrusion and which permit improved release of the active ingredient. Another object was to provide stable amorphous embedding of the active ingredients. The object of this invention also included improvement of the mechanical properties of the formulations.

The preparations defined in the introduction have accordingly been found. Processes for producing active-ingredient forms of this type have also been found.

The enlargement of the surface of the preparations takes place via partial or complete foaming of the preparation. “Partially or completely foamed” means in the invention that the foaming process achieves a density which is from 1% to 99% of the density of the compact preparation. The density produced is preferably from 2 to 50%. The foaming process can be assessed visually with the aid of optical or electron micrographs, or can be monitored via direct density determination. The term “foamed” is also used hereinafter as a synonym for “partially or completely foamed”.

The solid foamed active-ingredient preparations of the invention can comprise, as active ingredients, any of the substances which can be incorporated without decomposition into the polymer melt under the conditions of the process.

Suitable amphiphilic copolymers are polyether-containing graft polymers. These are obtained via free-radical polymerization of vinyl monomers in the presence of the polyether component, which serves as graft base.

Particularly suitable materials for producing the foamed preparations are polyether graft polymers which are obtained via free-radical-initiated polymerization of a mixture of
i) from 30 to 80% by weight of N-vinyllactam,
ii) from 10 to 50% by weight of vinyl acetate, and
iii) from 10 to 50% by weight of a polyether,
   with the proviso that the sum of i), ii), and iii) is 100% by weight.

In one variant of the process, the polyether copolymers are intimately mixed with polymers that are sparingly soluble in water and with the active ingredients that are sparingly soluble in water, and the mixture is heated above the glass transition temperature of the copolymers.

The polyether copolymers are readily soluble in water, and this means that 1 part of copolymer dissolves in from 1 to 10 parts of water at 20° C.

In one embodiment of the invention, preferred polyether copolymers are obtained from:
i) from 30 to 70% by weight of N-vinyllactam,
ii) from 15 to 35% by weight of vinyl acetate, and
iii) from 10 to 35% by weight of a polyether.

Polyether copolymers whose use is particularly preferred are obtainable from:
i) from 40 to 60% by weight of N-vinyllactam,
ii) from 15 to 35% by weight of vinyl acetate, and
iii) from 10 to 30% by weight of a polyether.

Polyether copolymers whose use is very particularly preferred are obtainable from
i) from 50 to 60% by weight of N-vinyllactam,
ii) from 25 to 35% by weight of vinyl acetate, and
iii) from 10 to 20% by weight of a polyether.

The proviso that the entirety of components i), ii), and iii) gives 100% by weight also applies to the preferred and particularly preferred constitutions.

The N-vinyllactam used can comprise N-vinylcaprolactam or N-vinylpyrrolidone, or a mixture thereof. It is preferable to use N-vinylcaprolactam.

Polyethers serve as graft base. Polyethers that can be used preferably comprise polyalkylene glycols. The molecular weights of the polyalkylene glycols can be from 1000 to 100 000 D [daltons], preferably from 1500 to 35 000 D, particularly preferably from 1500 to 10 000 D. The molecular weights are determined on the basis of the OH number measured to DIN 53240.

Particularly preferred polyalkylene glycols that can be used comprise polyethylene glycols. Other suitable materials are polypropylene glycols, polytetrahydrofurans, and polybutylene glycols, which are obtained from 2-ethyloxirane or 2,3-dimethyloxirane.

Other suitable polyethers are random or block-type copolymers of polyalkylene glycols obtained from ethylene oxide, from propylene oxide, or from butylene oxides, examples being polyethylene glycol-polypropylene glycol block copolymers. The block copolymers can be of AB type or of ABA type.

Among the preferred polyalkylene glycols are also those which have alkylation at one or both OH end groups. Alkyl radicals that can be used comprise branched or unbranched C₁-C₂₂-alkyl radicals, preferably C₁-C₁₈-alkyl radicals, such as methyl, ethyl, n-butyl, isobutyl, pentyl, hexyl, octyl, nonyl, decyl, dodecyl, tridecyl, or octadecyl radicals.

General processes for producing the polyether copolymers of the invention are known per se. The production process uses free-radical-initiated polymerization, preferably in solution, in nonaqueous, organic solvents, or in mixed nonaqueous/aqueous solvents. Suitable production processes have been described by way of example in WO 2007/051743 and WO 2009/013202, and the disclosure in these relating to the production process is expressly incorporated herein by way of reference.

The meaning of the expression “sparingly soluble in water” in the invention is as follows: in the invention, the expression “sparingly soluble in water” covers substances which are sparingly soluble to practically insoluble, and means that at least from 100 to 1000 g of water is required per g of substance in order to dissolve the substance in water at 20° C. In the case of practically insoluble substances, at least 10,000 g of water are required per g of substance.

The abbreviated term “sparingly soluble” is used hereinafter in the description to mean “sparingly souble in water”.

The present preparations have particular suitability for formulating pharmaceutical active ingredients, in particular for sparingly soluble active ingredients.

Examples of suitable active ingredients are: acebutolol, acetylcysteine, acetylsalicylic acid, aciclovir, alprazolam, albumin, alfacalcidol, allantoin, allopurinol, ambroxol, amikacin, amiloride, aminoacetic acid, amiodarone, amitriptyline, amlodipine, amoxicillin, ampicillin, ascorbic acid, aspartame, astemizole, atenolol, acemetacin, beclometasone, benserazide, benzalkonium hydroxide, benzocaine, benzoic acid, betametasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefadroxil, cefazolin, cefixime, cefotaxime, ceftazidine, ceftriaxone, cefuroxime, chloramphenicol, chlorhexidine, chlorpheniramine, chlortalidone, choline, ciclosporin, cilastatin, cimetidine, ciprofloxacin, cisapride, cisplatin, clarithromycin, clavulanic acid, clomipramine, clonazepam, clonidine, clotrimazole, clozapine, codeine, colestyramine, cromoglicic acid, cyanocobalamin, cyproterone, desogestrel, dexamethasone, dexpanthenol, dextromethorphan, dextropropoxiphene, diazepam, diclofenac, digoxin, dihydrocodeine, dihydroergotamine, diltiazem, diphenhydramine, dipyridamole, dipyrone, disopyramide, domperidone, dopamine, doxycycline, enalapril, enrofloxacin, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin mononucleotide, fluconazole, flunarizine, fluorouracil, fluoxetine, flurbiprofen, flutamide, furosemide, gemfibrozil, gentamicin, Ginkgo biloba, glibenclamide, glipizide, Glycyrrhiza glabra, guaifenesin, haloperidol, heparin, hyaluronic acid, hydrochlorothiazide, hydrocodone, hydrocortisone, hydromorphone, hydroxytetracycline, ipratropium hydroxide, ibuprofen, imipenem, indomethacin, iohexol, iopamidol, isosorbide dinitrate, isosorbide mononitrate, isotretinoin, ketotifen, ketoconazole, ketoprofen, ketorolac, labetalon, lactulose, lecithin, levocarnitine, levodopa, levoglutamide, levonorgestrel, levothyroxine, lidocaine, lipase, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamins and minerals, nystatin, N-methylephedrine, naftidrofuryl, naproxen, neomycin, nicardipine, nicergoline, nicotinamide, nicotine, nicotinic acid, nifedipine, nimodipine, nitrendipine, nizatidine, norethisterone, norfloxacin, norgestrel, nortriptyline, ofloxacin, omeprazole, ondansetron, pancreatin, panthenol, pantoprazole, pantothenic acid, paracetamol, penicillin G, penicillin V, phenobarbital, pentoxifylline, phenylephrine, phenylpropanolamine, phenytoin, piroxicam, polymyxin B, povidone-iodine, pravastatin, prazepam, prazosin, prednisolone, prednisone, proglumetacin, propafenone, propranolol, pseudoephedrine, pyridoxine, quinidine, ramipril, ranitidine, reserpine, retinol, riboflavin, rifampicin, rutoside, saccharin, salbutamol, salcatonin, salicylic acid, sildenafil, simvastatin, somatropin, sotalol, spironolactone, sucralfate, sulbactam, sulfamethoxazole, sulpiride, tamoxifen, tegafur, tenoxicam, teprenone, terazosin, terbutaline, terfenadine, theophylline, thiamine, tiaprofenic acid, ticlopidine, timolol, tranexamic acid, tretinoin, triamcinolone acetonide, triamterene, trimethoprim, troxerutin, uracil, valproic acid, vancomycin, verapamil, vitamin E, folinic acid, zidovudine, zotepine. Vitamins can also be formulated according to the invention. These include vitamins of the A group, of the B group, and therefore not only B1, B2, B6 and B12 and nicotinic acid and nicotinamide, and also compounds with vitamin B properties e.g. adenine, choline, pantothenic acid, biotin, adenylic acid, folic acid, orotic acid, pangamic acid, carnitine, p-aminobenzoic acid, myo-inositol and alpha-lipoic acid, and also vitamins of the C group, D group, E group, F group, H group, I and J groups, K group, and P group.

Other active ingredients that can be used are plant-protection agents, other biocides, or substances used in veterinary medicine.

The preparations can also receive additions of other thermoplastically processable polymers, alongside the amphiphilic copolymers.

Other thermoplastically processable polymers that can be used for the polymer matrix are amorphous, thermoplastic polymers of the invention.

Polymers that are especially suitable are water-soluble, thermoplastically processable homo- or copolymers of N-vinylpyrrolidone, or a mixture of said polymers. The glass transition temperatures of the polymers are usually in the range from 80 to 190° C., preferably from 90 to 175° C. Examples of suitable homopolymers are polymers with Fikentscher K values in the range from 10 to 30. Suitable copolymers can comprise, as comonomers, unsaturated carboxylic acids, e.g. methacrylic acid, crotonic acid, maleic acid or itaconic acid, or else esters thereof with alcohols having from 1 to 12, preferably from 1 to 8, carbon atoms, or else 25 hydroxyethyl or 25 hydroxypropyl acrylate and the corresponding methacrylates, (meth)acrylamide, the anhydrides and hemiesters of maleic acid and itaconic acid (where the hemiester is preferably not formed until after the polymerization reaction), or vinyl monomers, such as N-vinylcaprolactam, vinyl acetate, vinyl butyrate, and vinyl propionate, or else a mixture of these comonomers. By way of example, suitable materials are therefore terpolymers of N-vinylpyrrolidone, vinyl acetate, and vinyl propionate.

Acrylic acid is a preferred comonomer and vinyl acetate is a particularly preferred comonomer. The amounts comprised of the comonomers can be from 20 up to 70% by weight. Very particular preference in the invention is given to copolymers which are obtained from 60% by weight of N-vinylpyrrolidone and 40% by weight of vinyl acetate.

Other examples of suitable polymers are homo- or copolymers of vinyl chloride, polyvinyl alcohols, polystyrene, polyhydroxybutyrates, and copolymers of ethylene and vinyl acetate.

The active-ingredient preparations can moreover also comprise starches, degraded starches, casein, pectin, chitin, chitosan, gelatin, or shellac as matrix components, where these can be processed in the melt with addition of conventional plasticizers.

The preparations of the invention can moreover comprise the conventional pharmaceutical auxiliaries, such as bulking agents, lubricants, mold-release agents, flow regulators, plasticizers, dyes, and stabilizers in amounts of up to 50% by weight. These amounts, and the amounts stated hereinafter, are always based on the total weight of the preparation (=100%).

Examples of bulking agents that may be mentioned are the oxides of magnesium, aluminum, silicon, and titanium, and also lactose, mannitol, sorbitol, xylitol, pentaerythritol, and its derivatives, where the amount of bulking agent is in the range from 0.02 to 50% by weight, preferably from 0.2 to 20% by weight.

Examples that may be mentioned of flow regulators are the mono-, di-, and triglycerides of the long-chain fatty acids, such as C12, C14, C1S, and C1s fatty acid, waxes, such as carnauba wax, and also the lecithins, where the amount is in the range from 0.1 to 30% by weight, preferably from 0.1 to 5% by weight.

Examples that may be mentioned of plasticizers are not only low-molecular weight polyalkylene oxides, such as polyethylene glycol, polypropylene glycol, and polyethylene propylene glycol, but also polyfunctional alcohols, such as propylene glycol, glycerol, pentaerythritol, and sorbitol, and also sodium diethyl sulfosuccinate, glycerol mono-, di-, and triacetate, and polyethylene glycol esters of stearic acid. The amount of plasticizer here is from 0.5 to 15% by weight, preferably from 0.5 to 5% by weight.

Examples of lubricants that may be mentioned are stearates of aluminum or calcium, and also talc and silicones, where the amount of these is in the range from 0.1 to 5% by weight, preferably from 0.1 to 3% by weight.

Examples of stabilizers that may be mentioned are light stabilizers, antioxidants, free-radical scavengers, and stabilizers to counteract microbial infestation, where the amount of these is preferably in the range from 0.01 to 0.05% by weight.

To produce the preparations of the invention, the active-ingredient component can either be premixed with the polymer and then extruded or else can be fed to the polymer melt comprising blowing agent during the extrusion process.

The quantitative proportions of the individual components within the preparation can be varied within wide limits. As a function of active-ingredient dose and active-ingredient release rate, the amount thereof can be from 0.1 to 90% by weight of the active-ingredient preparation. The amount of the polymer can be from 10 to 99.9% by weight. The material can also comprise from 0 to 50% by weight of one or more auxiliaries.

Production of foamed active-ingredient preparations of the invention is preferably achieved via extrusion of a melt which comprises, alongside one or more active ingredients, at least one amphiphilic copolymer and also optionally further thermoplastically processable polymers and also optionally conventional auxiliaries, where the melt has been impregnated with volatile blowing agents that are physiologically acceptable.

Suitable volatile, physiologically nonhazardous blowing agents are gaseous blowing agents, such as carbon dioxide, nitrogen, air, inert gases, e.g. helium or argon, propane, butane, dimethyl ether, ethyl chloride, chlorofluorocarbons, difluoroethane, or dinitrogen oxide (laughing gas), preference being given to carbon dioxide and/or nitrogen. Carbon dioxide is very particularly preferred. The gaseous blowing agent can be used in subcritical state, but use in supercritical state is preferred.

The melt is preferably produced in an extruder, particularly preferably in a twin-screw extruder. The mixing of the active ingredient(s) with the polymers and optionally with further additions can take place prior to or after the melting of the polymers, by processes conventional in the art. Particularly when active ingredients are sensitive to temperature, it is advisable to add these only after the thermoplastic has been melted. The melt can be obtained at temperatures of from 20 to 200° C., preferably from 70 to 200° C., and the suitable temperature depends especially on the glass transition temperature of the polymers added. As a function of the mixture components present in the melt, the extrusion process can also be carried out at temperatures below the glass transition temperature of the amphiphilic copolymer. It is also possible that the blowing agent reduces the temperatures required for obtaining a melt, because it has a viscosity-reducing effect.

The polymers will usually be melted at temperatures above their glass transition temperature.

Impregnation of the melt with the blowing agent is preferably achieved under pressure. Under these conditions from 1 to 15% by weight of the blowing agent can dissolve in the melt. The gas can be introduced here at pressures of up to 30 MPa, preferably at pressures of from 0.1 to 20 MPa. Up to 20 percent by weight of gas are injected into the melt here—preferably from 1 to 20% by weight. The impregnation with plasticizing blowing agents, such as CO₂, reduces the viscosity of the melt, and the temperatures at which the melt comprising blowing agent can be extruded are therefore lower than those for a corresponding melt free from blowing agent. This property of the polymer melt comprising blowing agent is advantageous for the incorporation of thermally labile active ingredients.

Prior to extrusion through the die, the melt comprising blowing agent is cooled to temperatures in the range of up to 70° C. above the glass transition temperature of the mixture. The temperature at the die is preferably from 10 to 40° C. above the glass transition temperature of the formulation. A particularly preferred temperature range is from 15 to 30° C.

In the case of active ingredients that are particularly sensitive to temperature, it is advisable that addition to the melt takes place after admixture of blowing agent and after temperature reduction.

The process of the invention can be carried out in a single extruder with different temperature zones. However, preference is given to a tandem extrusion system composed of two extruders coupled to one another, where the first extruder, in which the melting of the polymer and the charging of blowing agent to the melt takes place, is preferably a twin-screw extruder with good mixing action, the second extruder being a single-screw extruder with little shearing action and high cooling capability.

The extrudate emerging from the extruder die remains plastic and expands under the atmospheric pressure prevailing outside of the extruder, to give a foam.

The amount of the blowing agent added, and the extrusion temperature, can be used to control the degree of foaming and therefore the morphology of the active-ingredient preparation.

A high degree of foaming gives a relatively low density and therefore a high rate of dissolution of the active-ingredient form. If relatively high densities are desired, a high blowing-agent content advantageous for the preparation can be lowered via devolatilization in the immediate vicinity of the die gap, thus giving a product which has been only slightly foamed. The foamed active-ingredient preparation is then subjected to a forming process to give the respective desired active-ingredient forms, for example by use of pelletization, granulation, or tableting, by known processes.

The densities of the solid active-ingredient preparations are usually in the range from 20 to 1000 g/I, preferably from 25 to 600 g/I, particularly preferably from 30 to 500 g/l.

The foams can be open-cell or closed-cell foams.

In comparison with conventional extrudates, the preparations of the invention have an enlarged surface.

It is also possible to use coextrusion to produce multilayered partially or fully foamed forms comprising active ingredients. Here, at least two compositions are coextruded and then subjected to a forming process to give the desired dosage form, and each composition comprises at least one of the abovementioned thermoplastic binders, and at least one of these compositions comprises at least one active ingredient, and at least one of these compositions has been impregnated in the manner described above with a gaseous physiologically nonhazardous blowing agent.

Prior to the coextrusion process, the composition for each layer of the active-ingredient form is prepared separately. To this end, the respective starting components are processed in a separate extruder to give melts comprising active ingredient, under the conditions described above for the above variant of the process. Operations for each layer here can be carried out under conditions that are respectively ideal for the specific material. By way of example, a different processing temperature can be selected for each layer. The respective compositions can by way of example also be impregnated with different amounts of blowing agent, thus producing layers with a different degree of foaming.

The molten or plastic compositions from the individual extruders are charged to a shared coextrusion die, and extruded and discharged. The shape of the coextrusion dies depends on the desired active-ingredient form. By way of example, suitable dies are those with a flat discharge gap, known as slot dies, and those with a discharge cross section in the shape of a circular gap.

The design of the die here depends on the polymeric binder used and on the desired shape.

After discharge from the coextrusion die, a forming process takes place to give the desired active-ingredient form or drug form. A wide variety of forms can be produced here, as a function of coextrusion die and of the nature of the forming process. By way of example, an extrudate emerging from a slot die and in particular having two or three layers can be used to produce open multilayer tablets by a punching or cutting process, for example using an incandescent wire.

As an alternative, open multilayer tablets can be produced by using a die with discharge cross section in the shape of a circular gap, with use of a die-face cutter, i.e. via chopping the extrudate immediately after discharge from the die, or preferably by use of a cold-cutting process, i.e. via chopping of the extrudate after at least some cooling.

Closed active-ingredient forms, i.e. forms in which the layer comprising active ingredient has been entirely surrounded by a layer free from active ingredient, are in particular obtained by using a die with discharge cross section in the shape of a circular gap, via treatment of the extrudate in a suitable nip device.

It is advantageous here if when the external layer has already cooled the internal layer of the multilayer tablet remains plastically deformable on entry into the nip device. This method can in particular be used to produce tablets, preferably oblong tablets, dragees, pastilles, and pellets.

In another variant of the process, foamed forms comprising active ingredient can be produced by extruding a melt which comprises not only one or more active ingredients but also a thermoplastic amphiphilic copolymer, subjecting the melt to a shaping process while it remains plastic, and then using one of the abovementioned gaseous blowing agents to impregnate the solid form comprising active ingredient, for example in a conventional autoclave at pressures in the range from 10 to 300 bar, preferably from 50 to 200 bar, and then expanding the material.

On depressurization to atmospheric pressure, the impregnated form expands to give a partially or fully foamed form.

The degree of foaming depends on the duration of the impregnation procedure and can be adjusted as desired. This variant of the process is preferably suitable for producing partially foamed forms which have an exterior foamed covering and an unfoamed core and therefore have a staged release profile.

The foamed forms can also be provided with a conventional coating that is permeable to active ingredient, thus giving easy access to floating forms. These floating forms can be utilized for pharmaceutical purposes or else for products used in veterinary medicine or in agricultural technology, an example being production of fish feed which sinks slowly.

The solid, foamed active-ingredient preparations obtained in the invention, and comprising the active ingredient homogeneously dispersed within the polymeric matrix, dissolve very rapidly and thus permit the rapid release of the active ingredient. The process of the invention can give the foamed active-ingredient preparations in a simple and cost-effective manner. Another advantage is that the viscosity-reducing effect of the blowing agent permits extrusion at temperatures markedly lower than those when blowing agent is absent, the result being less thermal stressing of the active ingredients.

The preparations of the invention have the active ingredient embedded in amorphous form. Amorphous means that no more than 3% by weight of the active ingredient, measured by DSC, is in crystalline form. The DSC measurements are usually carried out at a heating rate of 20 K/min.

The use of a solubilizing polymer here provides marked advantages in comparison with previously known polymers for the melt extrusion process. By virtue of the solubilizing effect, it is possible to achieve solid solutions even with active ingredients that have particularly low solubility. The relatively high specific surface area of the solid solutions obtained by the foaming process here provides a further increase in the rate of active-ingredient release in comparison with unfoamed solid solutions. Surprisingly, the foams produced with solubilizing polymers do not collapse even when relatively large amounts of lipophilic active ingredients are incorporated. This would have been an expected occurrence, since lipophilic additions, e.g. silicones, lead to drastic collapse of the foam structure in aqueous foams.

The gas used as temporary plasticizer during the extrusion process is no longer detectable in the final product, and it could therefore be assumed that stability is higher than in solid solutions with permanent plasticizers, since the latter preparations exhibit relatively low rigidity.

However, maximum rigidity is highly desirable in respect of the ease of milling of the foamed products. Products with relatively high stiffness are more brittle, and fracture more readily, and have less susceptibility to deformation during the grinding process. Retention of porous substructures is therefore better. The foamed preparations of the invention have good stiffness.

### EXAMPLES

The screw diameter of the twin-screw extruder used for producing the formulations described in the examples below was 16 mm and the length of the extruder was 40D. The entire extruder was composed of 8 individual temperature-controllable barrel sections. The first two barrel sections were temperature-controlled at 20° C. and, respectively, at 70° C., to improve material intake. A constant temperature was set at the third barrel section, and the gas input also took place here directly into the extruder via a die. The gas used comprised CO₂, input with the aid of a metering pump.

The temperature of the next barrel section was adjusted during the process so as to give a temperature at the die which can be up to 70° C. above the glass transition temperature of the mixture. After cooling, the foamed extrudate was milled for 30 s, using an analysis mill (IKA A10). For the examples below, the sieve fraction used was that which was smaller than 250 μm after grinding.

The crystalline or, respectively, amorphic nature of the resultant polymer foams were studied by means of XRD (X-ray diffractometry) and DSC (differential scanning calorimetry), with use of the following equipment and conditions:

### XRD

Measurement equipment: D 8 Advance diffractometer with 9-tube specimen changer (Bruker/AXS)

Type of measurement: θ-θ geometry in reflection

2 theta angle range: from 2 to 80°

Step width: 0.02°

Measurement time per angle step: 4.8 s

Divergence slit: Göbel minor with 0 4 mm inserted aperture

Antiscattering slit: Soller slit

Detector: Sol-X detector

Temperature: room temperature

Generator setting: 40 kV/50 mA

### DSC

DSC Q 2000 from TA Instruments

Parameters:

Weight used about 8.5 mg

Heating rate: 20 K/min

The ground foams were charged to hard gelatine capsules. A USP apparatus (paddle method) 2, at 37° C. and 50 rpm (BTWS 600, Pharmatest) in 0.1 molar hydrochloric acid for 2 h was used for active-ingredient release. UV spectroscopy (Perkin Elmer Lambda 2) was used to detect the active ingredient released. The specimens taken were diluted with methanol directly after filtration, in order to inhibit crystallization of the sparingly soluble active ingredient from the solution.

### Production of Polymer 1

The initial charge, without the partial amount of feed 2, was heated to 77° C. under N₂ in a stirred apparatus. Once the internal temperature had reached 77° C., the partial amount of feed 2 was added, and incipient polymerization was carried out for 15 min. Feed 1 was then metered in within a period of 5 h, and feed 2 within a period of 2 h. Once all of the feeds had been metered in, polymerization of the reaction mixture was continued for a further 3 h. After the continued polymerization process, the solution was adjusted to 50% by weight solids content.

Initial charge: 25 g of ethyl acetate

104.0 g of PEG 6000

1.0 g of feed 2

Feed 1: 240 g of vinyl acetate
Feed 2: 456 g of vinylcaprolactam

240 g of ethyl acetate

Feed 3: 10.44 g of tert-butyl perpivalate (75% strength by weight in aliphatic mixture)

67.90 g of ethyl acetate

The solvent was then removed via a spray process, and a pulverulent product was obtained. The K value, measured at 1% strength by weight in ethanol, was 16. The glass transition temperature of the polymer, determined by DSC, was 70° C.

### Example 1

### Polymer 1+5.5% by weight of CO₂

Throughput of polymer: 18.95 kg/h

CO₂ injection: 1.05 kg/h

Melt temperature prior to die: 135° C.

Melt pressure prior to die: 160 bar

### Example 2

### Polymer 1+8.4% by weight of CO₂

Throughput of polymer: 18.45 kg/h

CO₂ injection: 1.55 kg/h

Melt temperature prior to die: 120° C.

Melt pressure prior to die: 130 bar

### Example 3

### Polymer 1+11.1% by weight of CO₂

Throughput of polymer: 18 kg/h

CO₂ injection: 2 kg/h

Melt temperature prior to die: 107° C.

Melt pressure prior to die: 135 bar

### Example 4

### Polymer 1+fenofibrate+CO₂

Polymer 1 and 20% by weight of fenofibrate (melting point 81° C.), based on the entire amount of polymer+active ingredient, were weighed into a V blender and mixed for 60 minutes.
Throughput of mixture (polymer+fenofibrate): 18.75 kg/h
CO₂ injection: 1.25 kg/h
Melt temperature prior to die: 80° C.
Melt pressure prior to die: 135 bar

The foamed extrudates were studied by XRD and by DSC and found to be amorphous. Release of the active ingredient in 0.1 normal HCl after 2 h was 100%. The preparations remained amorphous after 6 months of storage at 30° C.

### Example 5

### Polymer 1+cinnarizine+CO₂

Polymer 1 and 20% by weight of cinnarizine (melting point 122° C.), based on the entire amount of polymer+active ingredient, were weighed into a V blender and mixed for 60 minutes.
Throughput of mixture (polymer+cinnarizine): 18.75 kg/h
CO₂ injection: 1.25 kg/h
Melt temperature prior to die: 105° C.
Melt pressure prior to die: 130 bar

The foamed extrudates were studied by XRD and by DSC and found to be amorphous. Release of the active ingredient in 0.1 normal HCl after 70 minutes was 95%. The preparations remained amorphous after 6 months of storage at 30° C.

### Example 6

### Polymer 1+itraconazole+CO₂

Polymer 1 and 40% by weight of itraconazole (melting point 166° C.), based on the entire amount of polymer+active ingredient were weighed into a V blender and mixed for 60 minutes.
Throughput of mixture (polymer+itraconazole): 18.9 kg/h
CO₂ injection: 1.1 kg/h
Melt temperature prior to die: 120° C.
Melt pressure prior to die: 130 bar

The foamed extrudates were studied by XRD and by DSC and found to be amorphous. Release of the active ingredient in 0.1 normal HCl after 2 h was 90%. The preparations remained amorphous after 6 months of storage at 30° C.

### Example 7

### Polymer 1+danazol+CO₂

Polymer 1 and 20% by weight of danazol (melting point 225° C.), based on the entire amount of polymer+active ingredient, were weighed into a V blender and mixed for 60 minutes.
Throughput of mixture (polymer+danazol): 18.75 kg/h
CO₂ injection: 1.25 kg/h
Melt temperature prior to die: 130° C.
Melt pressure prior to die: 135 bar

The foamed extrudates were studied by XRD and by DSC and found to be amorphous. Release of the active ingredient in 0.1 normal HCl after 2 h was 90%. The preparations remained amorphous after 6 months of storage at 30° C.

### Example 8

### Polymer 1+piroxicam+CO₂

Polymer 1 and 30% by weight of piroxicam (melting point 199° C.), based on the entire amount of polymer+active ingredient, were weighed into a V blender and mixed for 60 minutes.
Throughput of mixture (polymer+piroxicam): 18.85 kg/h
CO₂ injection: 1.15 kg/h
Melt temperature prior to die: 135° C.
Melt pressure prior to die: 140 bar

The foamed extrudates were studied by XRD and by DSC and found to be amorphous. Release of the active ingredient in 0.1 normal HCl after 2 h was 69%. The preparations remained amorphous after 6 months of storage at 30° C.

### Example 9

### Polymer 1+felodipine+CO₂

Polymer 1 and 40% by weight of felodipine (melting point 145° C.), based on the entire amount of polymer+active ingredient, were weighed into a V blender and mixed for 60 minutes.
Throughput of mixture (polymer+felodipine): 18.9 kg/h
CO₂ injection: 1.1 kg/h
Melt temperature prior to die: 110° C.
Melt pressure prior to die: 130 bar

The foamed extrudates were studied by XRD and by DSC and found to be amorphous. Release of the active ingredient in 0.1 normal HCl after 2 h was 91%. The preparations remained amorphous after 6 months of storage at 30° C.

### Example 10

### Polymer 1+carbamezipine+CO2

Polymer 1 and 40% by weight of carbamezipine (melting point 192° C.), based on the entire amount of polymer+active ingredient, were weighed into a V blender and mixed for 60 minutes.
Throughput of mixture (polymer+carbamezipine): 18.8 kg/h
CO₂ injection: 1.1 kg/h
Melt temperature prior to die: 130° C.
Melt pressure prior to die: 132 bar

The foamed extrudates were studied by XRD and by DSC and found to be amorphous. Release of the active ingredient in 0.1 normal HCl after 2 h was 78%. The preparations remained amorphous after 6 months of storage at 30° C.

### Example 11

### Polymer 1+clotrimazole+CO₂

Polymer 1 and 15% by weight of clotrimazole (melting point 148° C.), based on the entire amount of polymer+active ingredient, were weighed into a V blender and mixed for 60 minutes.
Throughput of mixture (polymer+clotrimazole): 18.7 kg/h
CO₂ injection: 1.3 kg/h
Melt temperature prior to die: 105° C.
Melt pressure prior to die: 135 bar

The foamed extrudates were studied by XRD and by DSC and found to be amorphous. Release of the active ingredient in 0.1 normal HCl after 2 h was 63%. The preparations remained amorphous after 6 months of storage at 30° C.

What is claimed is:

1. A foamed composition comprising an active ingredient and a thermoplastically processable amphiphilic copolymer, wherein the amphiphilic copolymer comprises at least one polyether-containing graft polymer.

2. The composition of claim 1, comprising a polyether graft polymer obtained by polymerization of N-vinyllactam, vinyl acetate and a polyether.

3. The composition of claim 2, wherein the polyether is a polyalkylene glycol.

4. The composition of claim 3, wherein the polyether is selected from the group consisting of polyethylene glycols, polypropylene glycols, polytetrahydrofurans, polybutylene glycols, and random or block-type copolymers of polyalkylene glycols.

5. The composition of claim 3, wherein the polyalkylene glycol is alkylated at one or both OH end groups.

6. The composition of claim 2, comprising a polyether graft copolymer obtained by free-radical-initiated polymerization of from 30 to 70% by weight of N-vinyllactam, from 15 to 35% by weight of vinyl acetate, and from 10 to 35% by weight of the polyether.

7. The composition of claim 6, comprising a polyether copolymer obtained by polymerization of from 40 to 60% by weight of N-vinyllactam, from 15 to 35% by weight of vinyl acetate, and from 10 to 30% by weight of a polyether.

8. The composition of claim 1, further comprising an amorphous, water-soluble thermoplastically processable polymer.

9. The composition of claim 8, wherein the amorphous, water-soluble thermoplastically processable polymer is selected from the group consisting of homo- or copolymers of N-vinylpyrrolidone; homo- or copolymers of vinyl chloride; polyvinyl alcohols; polystyrene; polyhydroxybutyrates; copolymers of ethylene and vinyl acetate; and mixtures thereof.

10. The composition of claim 9, wherein the amorphous, water-soluble thermoplastically processable polymer is selected from the group consisting of terpolymers of N-vinylpyrrolidone, vinyl acetate, and vinyl propionate.

11. A process for producing a composition according to claim 1, which comprises extruding and expanding a melt comprising the active ingredient, the at least one amphiphilic copolymer, and a volatile physiologically nonhazardous gas blowing agent.

12. The process of claim 11, wherein the melt further comprises an amorphous, water-soluble, thermoplastically processable polymer.

13. The process of claim 11, wherein the polyether graft polymer is obtained by polymerization of N-vinyllactam, vinyl acetate and a polyether.

14. The process of claim 11, wherein the melt is loaded with the blowing agent at a gas pressure of from 1 to 30 MPa.

15. The process of claim 11, wherein the concentration of the gas in the melt is from 1 to 15% by weight.

16. The process of claim 11, wherein the melt has a temperature of from 20 to 200° C.

17. The process of claim 16, wherein the melt has a temperature of from 70 to 200° C.

18. The process of claim 11, wherein the blowing agent is selected from the group consisting of carbon dioxide, nitrogen, air, inert gases, propane, butane, dimethyl ether, ethyl chloride, chlorofluorocarbons, difluoroethane, and dinitrogen oxide.

19. The process of claim 18, wherein the blowing agent is in a supercritical state.

20. The process according to claim 11, further comprising a process for forming the extruded and expanded melt.
